(19) United States
(12) Patent Application Publication
(10) Pub. No.: US 2021/0364664 A1
(43) Pub. Date: Nov. 25, 2021

(54) SCINTILLATOR PRODUCTS, APPARATUSES AND METHODS FOR USE IN AUTORADIOGRAPHIC IMAGING

(71) Applicant: Lightpoint Medical Limited, Waterside, Chesham (GB)

(72) Inventors: David Tuch, London (GB); Kunal Vyas, London (GB)

(73) Assignee: Lightpoint Medical Limited, Waterside, Chesham (GB)

(21) Appl. No.: 16/637,345

(22) PCT Filed: Aug. 3, 2018
(86) PCT No.: PCT/GB2018/052233

§ 371 Date: Feb. 7, 2020

(30) Foreign Application Priority Data
Aug. 9, 2017 (GB) 1712790.3

(51) Int. Cl. G01T 1/29; A61B 6/03; G01T 1/20; G01T 1/24

(52) U.S. Cl. CPC G01T 1/2942; G01T 1/24; G01T 1/2012; A61B 6/037

(57) ABSTRACT

Scintillator products, apparatuses and methods are provided for use in autoradiographic imaging of a tissue sample excised from a subject. In particular, scintillator products and devices are provided that are substantially conformable to a surface of the excised tissue sample and configured to scintillate, in use, in response to radiation from a radiopharmaceutical administered to the subject in advance of the excision.

### TECHNICAL FIELD

The present disclosure relates to scintillator products and apparatuses and methods of using said scintillator products for autoradiographic imaging. In particular, the present disclosure relates to scintillator products able to conform to the surface of a non-planar tissue sample, which may find a use in pre-clinical and clinical settings.

### BACKGROUND

If a patient is discovered to have an abnormal tissue growth, such as a cancerous tumour, then a surgeon may need to excise a tissue sample from the patient. This may be for the purpose of removing an entire tumour from the body, as part of a programme of therapy to stop further growth and spreading of the cancer. This is particularly the case for isolated tumours, but also those that have metastasised locally in the region of the originating tumour.

In the surgical removal of cancer, the surgeon is faced with many challenges and decisions. For example, when excising a tumour or other abnormality, a surgeon may need to decide on an amount of tissue containing the tumour to remove, with the aim of removal of the entirety of the tumour. However, when deciding how much tissue should be removed, there is a trade-off between removing as little tissue as possible to attempt to encompass the tumour without going beyond the margin of the tumour and into healthy tissue, and removing more than is necessary to ensure that the entire tumour has been removed from the patient yet causing collateral damage. Removing too much tissue can cause adverse post-operative effects for a patient. Currently a surgeon is guided as to the location, size and extent of a tumour by clinical diagnostic methods such as diagnostic scanning, for example, by ultrasound, mammography, Positron Emission Tomography (PET), Magnetic Resonance Imaging (MRI) scanning, performed on a patient in advance of a surgery. However, during surgery, a surgeon will make a judgement call based on experience and a tactile assessment of the excised sample to decide whether sufficient tissue has been removed to capture all of the tumour. If a surgeon is satisfied that the tissue excised is sufficient, the surgeon will close the incision and end the surgery.

Following the excision of the tissue sample, the patient is often sent home to recover while the tissue sample is sent to a pathology laboratory for histological analysis. The pathology lab may section or microtome the tissue sample and analyse the various tissue slices in order to determine whether or not the entire tumour has been excised. The microtoming and histological analysis takes days to weeks to complete, before an understanding of the extent and proliferation of the tumour is achieved.

Frequently it is discovered that the abnormal tissue or tumour is broaching the surface or is too close to the surface of the excised tissue sample to be confident that the entire abnormal tissue/tumour has been removed. That is, the histological analysis suggests that abnormal or cancerous tissue may have been left inside the patient, or the margins of tumour-free tissue towards the exterior of the tissue sample are too small to guarantee that all of the abnormal tissue or tumour has been removed from a patient. The patient may need to be recalled for reoperation in order to remove further tissue, which can be worrisome and unpleasant for the patient and requires further time and labour resources to be expended.

The present disclosure has been devised in the foregoing context.

### SUMMARY

In accordance with an aspect of the present invention there is provided a flexible scintillator product for use in autoradiographic imaging of a tissue sample excised from a subject. The flexible scintillator product comprises a membrane provided with a scintillator. The scintillator is configured to scintillate (luminesce), in use, in response to radiation from a radiopharmaceutical administered to the subject in advance of the excision. The membrane is freely conformable such that, in normal use, the scintillator product is wrappable around a surface of the excised tissue sample, for scintillation in response to radiation emitted therefrom.

Reference to a membrane may reference a film or a thin film structure. The membrane may have lateral dimensions much greater than its thickness. They membrane may be pliable or freely conformable and be in use not restricted from being able to be moved by wrapping around an excised tissue sample to assume a shape providing a surface contiguous with a surface of the excised tissue sample.

The flexible scintillator product may have a thickness of less than or equal to 3 millimetres. The scintillator product may have a thickness of less than or equal to 1 millimetre. The scintillator product may have a thickness of less than or equal to 500 micrometres. The scintillator product may have a thickness of less than or equal to 100 micrometres. The scintillator product may have a thickness of less than or equal to 20 micrometres. The scintillator product may have a thickness of less than or equal to 10 micrometres.

The flexible scintillator product may have a length less than or equal to 10 cm. The flexible scintillator product may have a breadth less than or equal to 10 cm.

The scintillator may comprise a polyvinyltoluene (PVT) based scintillator.

The membrane material may be the scintillator. For example, the membrane material may comprise a plastic scintillator such as BC-400, BC-404, BC-408, BC-498, or polyethylene naphthalate, “PEN”.

The scintillator may be integral to the membrane. For example, the scintillator may be dispersed in the material of the membrane during manufacture. For example, the scintillator may comprise a powdered scintillator. The scintillator may comprise ZnS:Ag, ZnCdS:Ag, YSO:Ce CsI:Tl, YAG:Ce, Y2O2S:Tb, or ZnSe:O.

The scintillator may be provided as a layer on the membrane. If the scintillator is provided as a layer on the membrane, a powdered scintillator may be used, for example, ZnS:Ag, ZnCdS:Ag, YSO:Ce CsI:Tl, YAG:Ce, Y2O2S:Tb, or ZnSe:O.

The scintillator product may be at least 10% transparent to electromagnetic waves having a wavelength in the range of 400 nm to 700 nm. That is, the scintillator product may be at least 10% transparent to visible light. The scintillator product may optionally be at least 20% transparent to visible light. The scintillator product may optionally be at least 30% transparent to visible light. The scintillator product may optionally be at least 40% transparent to visible light. The scintillator product may optionally be at least 50% transparent to visible light. The scintillator product may optionally be at least 60% transparent to visible light. The scintillator product may optionally be at least 70% transparent to visible light. The scintillator product may optionally be at least 80% transparent to visible light. The scintillator product may optionally be at least 90% transparent to visible light.

The membrane may comprise a plastic or elastomer substrate and a scintillator.

The excised tissue sample may comprise an intact excised tissue sample.

The scintillator may have a peak emission wavelength in the range of 400 nm to 500 nm. The scintillator may have a peak emission wavelength in the range of 410 nm to 450 nm. The scintillator may have a peak emission wavelength in the range 450 nm to 500 nm. The scintillator may have a peak emission wavelength in the range of 500 nm to 600 nm.

The scintillator product may further comprise a pattern, the pattern printed on the membrane and visible under illumination.

The scintillator may include a transparent dielectric or reflective coating for reducing losses. The transparent dielectric or reflective coating may be place on a surface of the scintillator product that is for contact with the excised tissue sample.

In accordance with an aspect of the present invention there is provided a scintillator product for use in autoradiographic imaging of a tissue sample excised from a subject. The scintillator product comprises a plurality of rigid scintillating elements. The plurality of rigid scintillating elements is configured to scintillate, in use, in response to radiation from a radiopharmaceutical administered to the subject in advance of the excision. The scintillating elements are moveable with respect to one another such that, in use, the scintillator product is substantially conformable to the surface of the excised tissue sample, for scintillation in response to radiation emitted therefrom.

The plurality of rigid scintillating elements may comprise a plurality of scintillating tiles. The scintillator product may comprise a mosaic of interlinked scintillating tiles. These scintillating elements may comprise a crystal scintillator such as CsI:Tl, GAGG:Ce, CdWO4, SrI2:Eu, CaF2:Eu, LaBr3:Ce, or a PVT-based or plastic scintillator such as BC-400, BC-404, BC-408, BC-498, or PEN.

The plurality of rigid scintillating elements may comprise a plurality of scintillating rods. Each scintillating rod may have a tip for contacting the excised tissue sample. The scintillator product may comprise a matrix of the scintillating rods. Each rod may be slideable through the matrix.

Each of the plurality of rigid scintillating elements may have a peak emission wavelength in the range of 400 nm to 500 nm. The scintillator may have a peak emission wavelength in the range of 410 nm to 450 nm. The scintillator may have a peak emission wavelength in the range 450-500 nm. Each of the plurality of rigid scintillating elements may have a peak emission wavelength in the range of 500 nm to 600 nm.

In accordance with an aspect of the present invention there is provided a scintillator product for use in autoradiographic imaging of a tissue sample excised from a subject. The scintillator product comprises a cloche shaped to form a cavity into which the excised tissue sample is to be received in use. The cloche thereby provides a covering for the excised tissue sample. The cloche is provided with a scintillator configured to scintillate in response to radiation from a radiopharmaceutical administered to the subject in advance of the excision. The scintillator may have a peak emission wavelength in the range of 400 nm to 500 nm. The scintillator may have a peak emission wavelength in the range of 410 nm to 450 nm. The scintillator may have a peak emission wavelength in the range 450 to 500 nm. The scintillator may have a peak emission wavelength in the range of 500 nm to 600 nm.

The scintillator products described herein may further comprise a pattern, the pattern printed on the membrane or the rigid scintillating tiles and visible under illumination, for example on a photographic image of the tissue sample.

The scintillator products described herein may be formed from polyethylene naphthalate, “PEN”. For example, the membrane or rigid scintillating elements may comprise polyethylene naphthalate.

A scintillator as described herein may comprise an inorganic compound such as Gadoliniumoxysulfide (Gd₂O₂S), also known as GOS or Gadox.

In accordance with an aspect of the present invention there is provided a collection of scintillator products as described herein. Each scintillator product of the collection of scintillator products may have a different shape and/or size.

In accordance with an aspect of the present invention there is provided a pack or dispenser storing a plurality of scintillator products as described herein. Each scintillator product of the plurality of scintillator products may be individually wrapped.

In accordance with an aspect of the present invention there is provided a scintillator device for use with a tissue sample excised from a subject, wherein in use a surface of the tissue sample is brought together with the scintillator device into a contiguous configuration in which the tissue sample and/or the scintillator device is deformed such that a surface of the tissue sample and a surface of the scintillator device substantially conform to one another. The scintillator device comprises a scintillator sheet configured to scintillate, in use, in response to radiation from a radiopharmaceutical administered to the subject in advance of the excision. The scintillator device further comprises a retention mechanism for retaining the scintillator sheet and the tissue sample together in the contiguous configuration.

The scintillator device may further comprise a positioning mechanism for bringing the tissue sample together with the scintillator device into the contiguous configuration.

The scintillator sheet may be rigid. In the contiguous configuration, the tissue sample may be deformed such that the surface of the tissue sample and the surface of the scintillator sheet substantially conform to one another.

The scintillator device may further comprise a transparent layer which, in the contiguous configuration, is positioned between the tissue sample and the scintillator. That is, the contiguous configuration may comprise a configuration in which the surface of the tissue is touching/in contact with the surface of the scintillator sheet. The contiguous configuration may comprise a configuration in which the surface of the tissue is touching/in contact with the transparent layer which is in turn touching the scintillator sheet.

In accordance with an aspect of the present invention there is provided a kit comprising a scintillator product as described herein and a set of instructions for wrapping an excised tissue sample in the scintillator product.

In accordance with an aspect of the present invention there is provided a kit for performing autoradiography of a tissue sample excised from a subject. The kit comprises a scintillator product as described herein and a detection apparatus. The detection apparatus comprises an enclosure for receiving the excised tissue sample with the scintillator product. The detection apparatus further comprises a detector for detecting scintillated light from within the enclosure.

In accordance with an aspect of the present invention there is provided a method for analysing a tissue sample excised from a subject. The method comprises providing a scintillator product as described herein to the tissue sample. The method further comprises detecting scintillated light from the scintillator product, the scintillated light generated in response to radiation emitted from the tissue sample.

The method may further comprise enclosing the tissue sample with the scintillator product in a light tight enclosure.

Detecting scintillated light may comprise detecting scintillated light using an electron multiplying charge coupled device, emCCD. Detecting scintillated light may comprise detecting scintillated light using a scientific complementary metal-oxide-semiconductor, sCMOS, detector.

The method may further comprise capturing an illuminated or photographic image of the tissue sample. Capturing an illuminated or photographic image of the tissue sample may comprise capturing a photographic image or illuminated image of the surface of the excised tissue sample. The method may further comprise mapping detections of the scintillated light to corresponding locations on the photographic image.

The method may further comprise determining, from the corresponding locations on the photographic image, whether the radiation was emitted from within a margin of a boundary of the tissue sample. The margin may be substantially 1 mm.

The scintillator product may further comprise a pattern, the pattern printed on a membrane or a rigid scintillating tile and visible on the photographic image of the tissue sample. The method may further comprise extracting a contour of the surface of the excised tissue sample from the pattern.

Detecting scintillated light may comprise detecting scintillated light using a plurality of spatially-separated detectors.

Providing a scintillator product to the tissue sample may comprise automatically providing a scintillator product to the tissue sample, or may comprise manually providing a scintillator product to the tissue sample.

According to an aspect of the present invention, a method is provided. The method comprises applying a scintillator product to a tissue sample excised from a subject. Applying the scintillator product to the tissue sample comprises deforming the tissue sample and/or the scintillator product such that a surface of the tissue sample and a surface of the scintillator product substantially conform to one another. The scintillator product is configured to scintillate, in use, in response to radiation from a radiopharmaceutical administered to the subject in advance of the excision.

The method may further comprise detecting scintillated light from the product, the scintillated light generated in response to the radiation emitted from the radiopharmaceutical administered to the subject in advance of the excision.

### BRIEF DESCRIPTION OF THE DRAWINGS

Embodiments of the invention are further described hereinafter with reference to the accompanying drawings, in which:

FIG. 1 shows a specimen imaging chamber containing a tissue sample wrapped in a flexible scintillator product in accordance with an embodiment of the invention;

FIG. 2 is a flowchart of a method for using a flexible scintillator product;

FIG. 3 shows a scintillator product in accordance with an embodiment of the invention;

FIG. 4 shows a scintillator product in accordance with an embodiment of the invention;

FIG. 5 shows a scintillator product in accordance with an embodiment of the invention;

FIG. 6 shows a scintillator device in accordance with an embodiment of the invention;

FIG. 7a shows a schematic of an experimental set up for investigating the effect of the spatial difference and angle between a radioactive source and a scintillator on image resolution;

FIG. 7b shows a photograph of the experimental set up of FIG. 7a;

FIG. 7c shows two images indicative of how the spatial resolution of the scintillated light images varies with the height difference between the radioactive source and a scintillating plate;

FIG. 8 shows a plurality of scintillated light images taken at different relative angles and heights between a radioactive source and a rigid scintillating plate;

FIG. 9 shows a photograph of a breast tissue phantom with radioactive sources applied;

FIG. 10 shows a plurality of scintillated light images using a Technetium-99m radioactive source that indicate that a flexible configuration provides better spatial resolution than a rigid configuration; and

FIG. 11 shows a plurality of scintillated light images using Fluorine-18 and Gallium-68 that indicate that a flexible configuration provides a better spatial resolution than a rigid configuration.

Throughout the description and drawings, like reference numerals refer to like parts.

### DETAILED DESCRIPTION

The present disclosure is concerned with improved methods and apparatus for optical imaging of radiopharmaceuticals that are more practical for specimen imaging in clinical settings and/or provide images that are more useful for clinical analysis.

Although in what follows, reference is made to clinical operations, and in particular to the excision of abnormal tissue including a tumour from a subject or patient in a clinical setting, the products, apparatuses, and methods described herein may be used in other clinical settings, or in pre-clinical research, or in veterinary treatment and the skilled person will appreciate that the detailed description that follows is not intended to limit the many and varied applications for which the products, apparatuses and methods described herein may be used.

The inventors have recognised that reoperation of patients due to unintentionally insufficient excision of tissue can be reduced if practical means and methods are developed to evaluate the abnormality/tumour-free margins of an excised tissue sample more quickly and efficiently, for example while in the operating theatre during an on-going surgical procedure. In particular, during a procedure to remove abnormal (e.g. cancerous) tissue from a subject it would be very beneficial for a surgeon to be able to confirm prior to conclusion of the procedure that they have removed all of the abnormal tissue. Accordingly, in such a situation, if it cannot be determined that all of the abnormal tissue (which may be, for example, a tumour) has been removed, for example by determining that the abnormal tissue is too close to the surface of the excised sample to be confident that all of the tumour has been removed, then the surgeon is able to excise a further tissue sample, ideally targeted at the remaining abnormal tissue, in the operating theatre and thereby greatly reduce the likelihood that reoperation is required.

One of the problems associated with performing an analysis of the tumour-free margins of an excised tissue sample in the operating theatre is that the excised tissue sample may be amorphous or possess an otherwise irregularly defined shape. One way of analysing the excised tissue sample would be to section the tissue sample, much as would often happen in a pathology laboratory, in order to analyse individual slices, and then to evaluate the individual slices. However, preparing and sectioning the sample, and subsequently analysing each slice of the sectioned sample is a time-consuming process and difficult to do efficiently and accurately in an operating theatre. Furthermore, it is not clear how one would analyse and keep track of individual slices of a sample in an operating theatre setting.

The inventors have further recognised that there are benefits to being able to analyse an amorphous or intact tissue sample to determine whether or not the margins towards the exterior of the sample are sufficient to ensure that the tumour has been removed. In particular, by being able to analyse an amorphous or intact tissue sample, the time and resources required to analyse the sample are greatly reduced. Products and methods suitable for this purpose are detailed herein.

The inventors have further recognised that one way of performing said analysis is to administer a radiopharmaceutical to the patient or subject in advance of the operation, the radiopharmaceutical for indicating the location of the tumour in the body. A radiopharmaceutical is a drug that can be used for diagnostic or therapeutic purposes and comprises a radioisotope bonded to a molecule. The radiopharmaceutical conveys the isotope to specific organs, tissues or cells. The radiopharmaceutical is selected for its properties and purpose. Many radiopharmaceuticals are known in the art and are used for radioguided surgery and other procedures. The radionuclides can usually be categorised by their decay modes, namely alpha decay, beta decay (electrons or positrons), electron capture and/or isomeric transition. Some beta decaying radioisotopes, including Fluorine-18 (¹⁸F), Carbon-11 (¹¹C), Nitrogen-13 (¹³N), Copper-64 (⁶⁴Cu), Iodine-124 (¹²⁴I) and Gallium-68 (⁶⁸Ga), emit positrons during radioactive decay and are known to be used in positron emission tomography (PET) imaging. Some beta decaying radioisotopes, including tritium (³H), Carbon-14 (¹⁴C), and Silicon-35 (³⁵S), emit electrons during radioactive decay.

Beta decay in the form of electrons or positrons can usually only penetrate a few millimetres through tissue, and so if beta radiation can be detected from an amorphous or intact sample, then a determination can be made that the radiation originated from within a few millimetres' depth from a surface of the sample, which can imply that the tumour-free margins in the excised tissue sample are not sufficient to indicate that the tumour has been fully removed.

The inventors have realised that, during surgery, the technique of autoradiography could be useful in imaging the distribution of radioactive particles emitted by the radioisotope used as a molecular tracer of the abnormal tissue in the excised tissue sample.

An autoradiograph or autoradiogram is an image, often on an x-ray film or nuclear emulsion, produced by the pattern of decay emissions (for example, beta particles or gamma rays) from a distribution of a radioactive substance in a tissue sample. Another method for performing autoradiography is to employ a scintillator. A scintillator is a material that exhibits scintillation—the property of luminescence, when excited by ionizing radiation. Luminescent materials, when struck by an incoming particle, absorb the energy of the particle and re-emit the absorbed energy in the form of light, which can be detected using imaging means such as will be described herein. Imaging such scintillation events using a digital imaging means comprising a sensitive surface such as a CCD provides a digital autoradiographic image. To provide a result useful to a surgeon, autoradiography of the excised tissue sample needs to provide an image with sufficient spatial resolution.

As will be discussed below, a radiopharmaceutical may be administered to the patient in advance of the operation to locate a tumour in the body of the patient. In accordance with examples of the present disclosure, once a tissue sample has been excised, the tissue sample may be placed within a dark or light tight enclosure of an imaging means as described further below.

The inventors have realised that, if, for example, a rigid scintillator plate, were suspended between the sample and an imaging means to form an autoradiographic image the tissue sample using with the imaging means, the image obtained by the imaging means would not reveal useful results for the following reasons. A separation distance between a radiation source in a sample and a scintillator leads to a poor spatial resolution as there is a large distribution of locations on the scintillator of incident radiation from the same point source. This leads to a distribution of scintillation events across an area of the scintillator from the same point source. Further, as the scintillation events are spread across a wider area, the imaged radiographic signal strength from radioactive decay events at the same point source of the sample leads to a lower signal to noise ratio and requires a longer integration time. The quality of autoradiographic imaging of intact tissue samples is rendered further unusable as the surface of the sample is not flat, leading to variations in the distance between the surface of the sample and the rigid scintillator plate. Thus autoradiographic imaging of an intact tissue sample using a rigid scintillator suspended above the sample would give a non-uniform spatial resolution and signal to noise resolution across the autoradiographic image of the sample.

Accordingly, when a rigid scintillator plate is used for autoradiographic imaging of an excised tissue sample in the manner described above, on detection of a scintillation event (by detecting light emitted from the scintillator plate) it is difficult to determine from where within the tissue sample the radiation originated, thereby making it difficult to determine whether the margins of the tissue sample are large enough to guarantee that the tumour has been removed.

One way of addressing this would be to bisect the sample to provide a flat surface, above which a rigid scintillator plate could be closely suspended. However, this would then only enable autoradiographic imaging of one section of the inside of the sample, rather than around the surface of the excised sample, and so this could not provide useful information for margin assessment by a surgeon during surgery.

The inventors have recognised that a scintillator product able to substantially conform to a surface of an excised tissue sample can greatly improve the spatial resolution and signal strength against a noise background of any autoradiographic image derived from the imaging means, as the distance between any given point on the surface of the sample and a corresponding point on the scintillator product can be substantially uniform. Several such scintillator products are described in detail herein. The inventors have further recognised that if one is able to manipulate the tissue sample itself in such a way that, without damaging the sample, the surface of the sample can be made to conform to a surface of a scintillator product or device, then similar benefits in spatial resolution and signal strength are achieved.

Beta decay in the form of electrons or positrons can usually only penetrate a few millimetres through human tissue. Advantageously, this means that if a scintillator product such as the scintillator products described herein is used and no scintillation events are detected by imaging means then a determination may be made that no radioactive markers are present within a few millimetres' depth of the surface of the tissue. This information can in turn be used to conclude that there is a margin of a few millimetres' depth from the surface of the sample in which there is no tumour present and it is likely that the entire tumour has been removed from the subject.

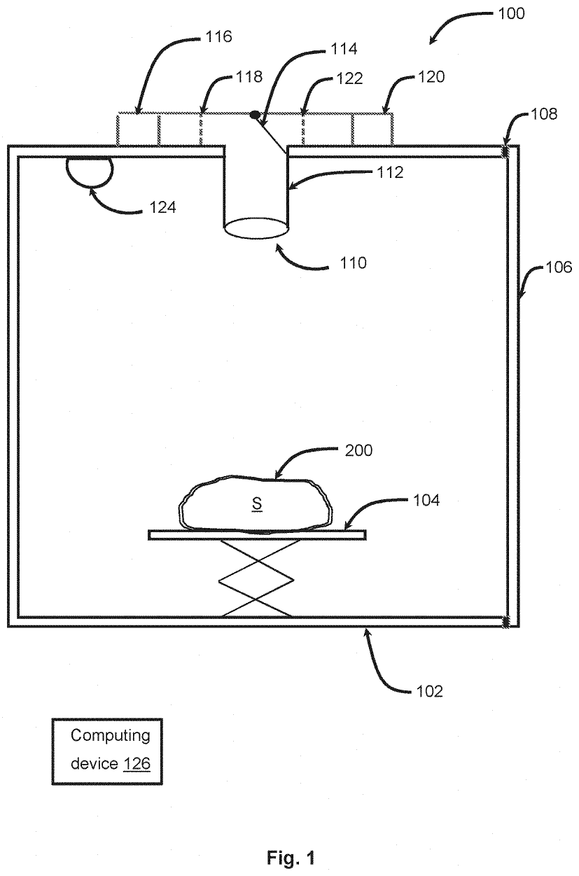

FIG. 1 shows an imaging apparatus 100 which can be used to image an object, for example a tissue sample. The skilled person will appreciate that the imaging apparatus may be suitable for imaging other objects. The skilled person will also comprehend that the imaging apparatus 100 of FIG. 1 is described as an example only and that other architectures are available.

The apparatus 100 is suitable for use by a surgeon or nurse or other medical professional in a clinical setting. The apparatus 100 includes a light tight chamber/enclosure 102 in which a sample S can be supported on a sample platform 104. The sample S is wrapped in a scintillator product 200 according to a first embodiment of the invention as will be described in detail further below. The sample platform 104 may be raised or lowered in order to alter the height of the sample S within the chamber—altering the height of the platform can improve the quality of the resultant images. The light tight enclosure 102 has a door 106 that can be opened to give access to the interior of the enclosure 102, for example, for introduction or removal of the sample S. A seal 108 around the periphery of the door 106 ensures the light tightness of the chamber 102 when the door 106 is closed.

An imaging system is mounted towards the top of the apparatus 100. This system comprises one or more optical components 110, for example a lens, that captures light from within the enclosure 102 and passes the light along a light tight optical conduit 112. The light tight optical conduit 112 contains a double-sided mirror 114, pivotably moveable relative to the internal wall of the optical conduit 112 by, for example, a hinge mounting, and configured to move between a first position, in which light is directed from the lens to a first imaging means 116 via a first optical filter 118, and a second position in which light is directed from the lens to a second imaging means 120 via a second optical filter 122. In the present example, the first imaging means 116 comprises a camera and the second imaging means 120 comprises a camera. The one or more optical components may, optionally together with the first 116 and second 120 imaging means, form an image of the sample S for capture by the first 116 and second 120 imaging means.

The set-up shown in FIG. 1 allows both imaging means 116 and 120 to be at least partially radiation shielded by the enclosure 102. The enclosure 102, for example the walls of the enclosure 102 or sections thereof, may be provided with or formed of a material, such as lead, that is highly attenuating to radiation emitted from sample S. This reduces the background noise in the images captured by imaging means 116, 120. The set-up further allows for the surfaces of each image detector/camera 116, 120 to be normal to the top face of the chamber 102 on which each image detector/camera is supported so as to minimise the cross-section of the detector potentially exposed to x-rays or beta-particles escaping the chamber. The configuration of the double-sided mirror 114 allows for some light to be directed to the first detector 116 and for some light to be directed to the second detector 120 depending on the angle and/or positioning of the mirror. Alternatively, a beam splitter arrangement may be used. The imaging means 116 and 120 both image light from the sample S along the same effective light path, allowing an overlay of image of the same region of the surface of the sample S to be achieved with positional correspondence.

The imaging apparatus 100 further comprises a light source 124 mounted inside the light tight enclosure. The light source 124 is for illuminating the interior of the enclosure 102 with white light or RGB light, which can be used to help directly image the sample S. The light source may comprise a light emitting diode (LED). By illuminating the interior of the enclosure, the first imaging means 116 is able to capture an image of the physical structure of the sample S.

Each of the mirror 114, the first imaging means 116, the second imaging means 120 and the light source 124 are communicatively coupled to a computing device 126. The computing device comprises a processor and a memory, the processor configured to execute instructions stored on the memory or input through a user input device.

In use, a sample S may be provided with a scintillator product such as scintillator product 200 and placed within the light tight enclosure 102 whereupon the door to the light tight enclosure 106 is subsequently closed. The computing device 126 configures the mirror 114 in the first position such that any light from the lens 110 is filtered by the first filter 118 and impinges upon the first camera 116. The computing device 126 uses the light source 124 to illuminate the interior of the enclosure 102. Accordingly, the first imaging means 116 captures an illuminated image of the sample S. The first imaging means 116 communicates the illuminated image to the computing device 126. The computing device 126 then switches off the light source 124 such that the enclosure 102 is in darkness and configures the mirror 114 to the second position such that any light received from the lens 110 is directed to the second imaging means 120. The second imaging means 120 communicates any detected image to the computing device 126. In particular, if the sample S is provided to the enclosure 102 with a scintillator product 200 as described herein, then the second imaging means 120 will detect scintillation events and communicate the low light luminescence image to the computing device 126. A longer exposure and integration time may be needed to capture the autoradiographic image of the sample from imaging means 120, than the illuminated image of the sample using imaging means 116.

The second optical filter 122 is configured to optimise detection (by the second imaging means 120) of any scintillation events that occur within the light tight enclosure 102. Accordingly, the second optical filter may be optimised for filtering out wavelengths that are not in the range of 500 nm to 600 nm. The second camera 120 in FIG. 1 comprises an electron multiplying charge coupled device (emCCD) to acquire low light images.

The skilled person would understand that the imaging apparatus of FIG. 1 is purely illustrative and that other variations of the imaging apparatus would also be suitable. For example, the emCCD camera may be replaced with a scientific complementary metal-oxide-semiconductor (sCMOS) detector. The double-sided mirror may be replaced with some other filtering mechanism for directing illuminated images to the first imaging device 116 and low light images to the second imaging device 120. Alternatively, a single camera such as an emCCD may be used to collect both the illuminated image and the low-light image, or alternatively in some embodiments the illuminated image need not be acquired at all. The imaging apparatus may comprise a light tight enclosure and an imaging means, or may comprise further components.

The computing device 126 is configured to receive the illuminated image from the first imaging means 116 and to receive the low light luminescence image from the second imaging means 120, to process the images and, in the present example, to superimpose the processed images to provide an indication as to whether any scintillation events have been detected that correspond with a radiopharmaceutical being present within a predetermined distance from the surface of the sample S. By providing the sample S with a scintillator product in accordance with examples of the present disclosure, autoradiographic imaging of the scintillator product can reveal, with positional accuracy and strong signal to noise, the presence or absence of a sufficient margin around abnormal tissue in an intact excised sample.

As described above, it can be beneficial to be able to accurately determine the distribution of a radiopharmaceutical within an excised tissue sample and in particular whether the radiopharmaceutical is located within a predetermined distance from the surface of the tissue sample. Accordingly, it is beneficial to be able to correlate a scintillation event (wherein a scintillator product emits light in response to an interaction with impinging radiation from the sample) to a radiation origin within the sample.

The scintillator product 200 shown in FIG. 1 is now described. The sample S may be provided with the scintillator product 200 to the imaging apparatus 100 of FIG. 1 for performing autoradiographic imaging of the sample S.

The flexible scintillator product 200 is suitable for use in autoradiographic imaging of a tissue sample. The scintillator product 200 comprises a substrate or membrane provided with a scintillator. The scintillator is configured to luminesce in response to radiation impinging upon the scintillator product 200. The membrane may be very thin, such as a film.

The membrane is freely conformable such that, in normal use, the scintillator product 200 is wrappable around a surface of a tissue sample, for scintillation in response to radiation emitted therefrom.

The terms “wrappable”, “wrap”, and “wrapping” around a surface of a tissue sample as used herein are understood to mean substantially following the contours of the surface. The flexible scintillator product 200 may be used to wrap the entire sample such that all surfaces of the sample are completely covered by the scintillator product, or the scintillator product 200 may be used to wrap (i.e. substantially conform to) a surface or part of a surface of the tissue sample. The scintillator product 200 is flexible and not a rigid scintillator plate.

The scintillator product 200 may be layered, for example the scintillator may be vapour deposited onto the membrane, or may be formed within or intrinsically part of the membrane. In the present example, the scintillator product 200 comprises a phosphor that has been vapour deposited onto the membrane, the membrane comprising a flexible polymer substrate. However, the skilled person will appreciate that other methods of manufacture are available. A scintillator product as described herein may be manufactured by physical or chemical vapour deposition. A scintillator product as described herein may be manufactured using spin-coating techniques and in particular spin-coating of an elastomer loaded with scintillator powder. A scintillator product as described herein may be manufactured by casting of an elastomer loaded with scintillator powder. A scintillator product as described herein may be manufactured from extruded plastic with scintillator dopants.

The scintillator is chosen for the particle of interest. For example, for imaging Fluorine-18 positrons, the scintillator is chosen so as to be sensitive to positrons but insensitive to annihilation photons and low-energy Compton photons. For example, the sensitivity to beta radiation may be greater than or equal to 10⁴ photons per mega-electronvolt (ph/MeV).

Factors for selecting a scintillator for use comprise the intrinsic light yield from the scintillator, the transparency of the scintillator (in conjunction with the membrane), the amount of backscattering, uniform particle size and dispersibility. It may also be useful to select a scintillator for low density in order to ensure that the signal from beta particle scintillation events is stronger than the signal from gamma radiation scintillation events. It may also be desirable to consider the spectral overlap of the emission peak of the scintillator with the imaging detection apparatus. The scintillator is also chosen such that the overlap of the emission peak of the scintillator with the spectrum from artefactual sources such as tissue autofluorescence, Cerenkov luminescence, and chemiluminescence is not so great as to degrade the reliability of the detected image. For example, a scintillator may have an emission peak in the range of 400 nm to 500 nm, and in particular in the range of 400 nm to 450 nm or in the range of 450 nm to 500 nm. A scintillator may have an emission peak in the range of 500 nm to 600 nm.

The scintillator product 200 is largely transparent to visible light. For example, the scintillator product may provide greater than or equal to 60% transmission over the 400 nm to 700 nm range of wavelengths, or may provide greater than or equal to 10% transmission over the 400 nm to 700 nm range of wavelengths.

The thickness of the membrane is selected so as to capture a substantial portion of the beta radiation emitted from the sample, while not being so thick as to be overly sensitive to other emitted particles. The thickness of the membrane is also chosen to provide the scintillator product 200 with a high degree of flexibility so as to be substantially freely conformable to a surface of a tissue sample. For some purposes, a suitable thickness for the membrane may be around 3 mm, or around 1 mm, or around 0.5 mm. For some purposes, a suitable thickness for the scintillator product is around 20 μm (that is, the membrane is like a thin film). Through experimentation, the inventors have found a suitable thickness for the scintillator product to be around 12 μm. For a scintillator product having a thickness of around 12 μm, the membrane/film may have a thickness of around 6 μm and the scintillator may have a thickness of around 6 μm. The skilled person will appreciate that the scintillator product may have a different thickness from 12 μm, for example the scintillator product may have a thickness of around 6 to 10 μm. The skilled person would also appreciate that the term “thickness” of the scintillator product as used herein may or may not refer to a thickness of the scintillator product across the entire length and breadth of the scintillator product—the scintillator product may vary slightly across its length and breadth. Such variations may be due to the manufacture process.

The length and breadth of the scintillator product 200 may be of any suitable size. For example, for many purposes a length of between 80 and 100 mm is suitable for wrapping around a surface of a tissue sample. For example, a length of around 95 mm and a breadth of around 95 mm are suitable for being able to wrap a surface of a tissue sample. However, the skilled person will appreciate that the length and breadth of the membrane may take any other dimension. For example, the scintillator product 200 may be provided as a large sheet or roll from which shapes may be cut as required. Alternatively, the scintillator product 200 may be provided as an individual, pre-prepared and pre-sized form. The skilled person would further appreciate that the scintillator product 200 may not have a well-defined length or breadth, for example, the scintillator product 200 may be round in shape. The scintillator product 200 may be enclosed in a sterile package (not shown) which a clinician or automated wrapping or applicator machine may open to access the scintillator product for wrapping around the tissue sample. Plural scintillator products 200 may be provided in a pack or stack of scintillator products wrapped individually or together. The plural scintillator products may be provided in a dispenser arranged to dispense individual scintillator products for a clinician or applicator machine.

The membrane material may be the scintillator. For example, the membrane may comprise a plastic scintillator, such as BC-400, BC-404, BC-408, BC-498, or PEN.

Alternatively, the membrane may include a scintillator in powdered form. Any suitable scintillator may be used. For example, the scintillator may comprise Thallium activated Caesium Iodide, CsI(Tl). The scintillator may comprise ZnSe:O. The scintillator may comprise Cadmium Tungstate, CdWO4. The scintillator may comprise Gadolinium oxysulfide, GOS:Tb. The scintillator may have a low phosphorescence in response to visible light exposure in order to reduce the amount of background noise picked up by the detector.

The scintillator product 200 may be non-hygroscopic and/or non-permeable in order to reduce the likelihood of degradation of the scintillator product 200 over time.

The scintillator product 200 may further comprise a transparent dielectric layer and/or a reflective coating on the side of the scintillator product that is closest to the tissue sample in use. The effect of said dielectric layer or said reflective coating is to reduce losses due to backscattering in use.

Scintillated light may be directed towards the edge(s) of the scintillator product 200 due to total internal reflection, which may reduce the scintillation yield and create a halo artefact. This can be mitigated by providing a mask around the edges of the scintillator product 200.

The scintillator product 200 may further comprise a pattern visible on (for example, printed onto) the surface of the scintillator product which is primarily visible only on a photographic image of the illuminated sample. The pattern may comprise a grid printed on the scintillator product using an ink whose emission or absorption spectrum does not substantially overlap with the scintillation peak/emission peak of the scintillator product. When imaging, the three-dimensional contour of the surface of the sample can then be extracted from the deformed pattern using image processing procedures. Alternatively, the contour can be extracted using structured illumination.

The scintillator product 200 may comprise one or more further membrane layers provided with a scintillator. In this way, background gamma radiation noise may be suppressed. The scintillator product may comprise a hatching of a least two thicknesses in order to suppress the background noise from gamma radiation.

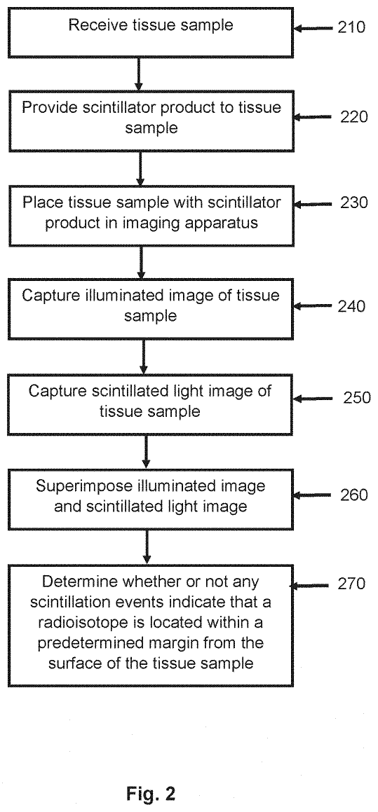

A method of using the scintillator product 200 of FIG. 1 is now described with reference to FIG. 2.

At step 210, a tissue sample is received, the tissue sample excised from a subject, the subject having been administered a radiopharmaceutical compound in advance of the excision.

At step 220, the scintillator product is provided to the tissue sample. In the example scintillator product 200 of FIG. 1, a surface of the tissue sample is wrapped in the scintillator product 200. As described above, the entire sample may be wrapped in the scintillator product 200 or the scintillator product 200 may be provided to a surface of the tissue sample S. The wrapping of the surface of the tissue sample may be performed manually or may be performed automatically for example, by an automated wrapping machine which may be provided within the imaging apparatus 100.

At step 230, the tissue sample is placed inside a substantially light tight enclosure of an imaging apparatus, such as that described above in relation to FIG. 1. The placement of the sample S is such that the wrapped surface of the sample will be imaged by the first imaging means 116 and the second imaging means 120. The sample S may be placed in the light tight enclosure 102 either manually or automatically.

At step 240 an illuminated image of the tissue sample S is captured. That is, the light source 124 of the imaging apparatus 100 is on such that the interior of the light tight enclosure 102 is illuminated and the light is directed from within the enclosure, via the light tight optical conduit, to the first imaging means 116. In this way, an illuminated image is captured from which information concerning the structure and/or shape of the tissue sample can be determined.

At step 250 a scintillated light image is detected. In particular, the light source 124 inside the enclosure 102 is turned off such that the interior of the light tight closure 102 is substantially in darkness. Radiation emitted from the sample S impinges upon the scintillation product 200 which in turn luminesces. The luminescence from the scintillator product 200 is directed to the second imaging means 120. It may be that no scintillation events occur, in which case the scintillated light image will not indicate any scintillation events. The scintillator product 200 is located close to the surface of the excised, intact sample S, around the visible surface of the sample, such that a distance between the sample and scintillator gives a high and uniform spatial resolution and high and uniform signal to noise in the autoradiographic image of the scintillation events in the scintillator product 200. Both the illuminated image and the scintillated light image are communicated to the computing device 126.

At step 260, the illuminated image and the scintillated light image are processed and superimposed such that, if any scintillation events have been detected, then at least one of the detected scintillation events is mapped to a position on the illuminated image. In this way, one may more closely determine from where within the sample S the radiation is being emitted.

At step 270, a determination is made as to whether or not any scintillation events indicate that a radiopharmaceutical is located within a margin of sensitivity from the wrapped surface of the tissue sample, S, the margin of sensitivity depending on the scintillator and radioisotope, but typically being on the order of ˜1-2 mm. For example, if no scintillation events are detected then it may be determined that no radionuclides are present within the margin of sensitivity from the surface of the sample. Alternatively, one may determine whether the margins are clear based on a quantity of scintillation events or other criteria.

The sample S may be removed from the imaging apparatus 100 manually or automatically. Further surfaces of the sample S may also be imaged. When multiple images of the sample S are captured, a three-dimensional image of the tissue sample (and radiation distribution within the tissue sample) may be generated.

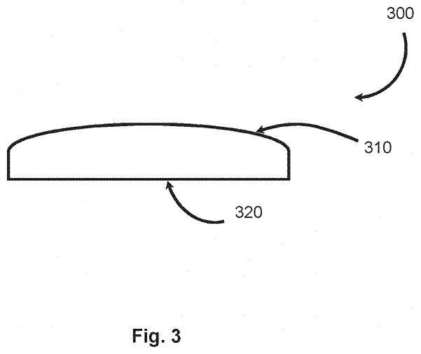

FIG. 3 illustrates a scintillator product according to another embodiment. In particular, FIG. 3 shows a scintillator product 300 for use in autoradiographic imaging of a tissue sample excised from a subject. The scintillator product 300 comprises a cloche 310 shaped to form a cavity 320 into which the excised tissue sample is to be received in use. The cloche 310 thereby provides a covering for the excised tissue sample. The cloche 310 is provided with a scintillator configured to scintillate in response to radiation from a radiopharmaceutical administered to the subject in advance of the excision. In use, a tissue sample may be provided to the cavity 320 of the scintillator product 300, and the scintillator product 300 is arranged to scintillate if radiation from the tissue sample in the cavity impinges upon the scintillator product 300. The curvature of the cloche 310 ensures that the scintillator product better matches the shape of a tissue sample than can be achieved with a rigid flat scintillator plate, and accordingly provides for better spatial resolution of any detected scintillation events.

As with the embodiment described above in relation to the scintillator product 200, the scintillator may be formed as a layer upon the cloche 310 by, for example, vapour deposition or spin coating. The scintillator may be integral to the cloche structure.

The cloche 310 may be rigid or may be deformable to adapt to a tissue sample provided within the cavity 320. The cloche 310 serves to provide a non-planar structure into which a tissue sample can be provided, so as to provide better spatial resolution of detected scintillation events than can be achieved using a flat rigid scintillation plate.

Although the cloche 310 of FIG. 3 is dome shaped or hemispherical, the cloche 310 (and the cavity 320 therein) may be of any suitable size and shape. For example, the cloche 310 may be cylindrical, cubic, conic or formed of any other suitable shape, such as a tetrahedron. A kit of plural cloches of a variety of shapes and sizes may be provided, such that the cloche having a size and shape most suitable for an excised tissue sample may be selected.

The scintillator of the embodiment of FIG. 3 is any suitable scintillator and can be selected based on the same considerations as those for selecting the scintillator of scintillator product 200. Any suitable scintillator may be used. For example, the scintillator may comprise Thallium activated Caesium Iodide, CsI(Tl). The scintillator may comprise ZnSe:O. The scintillator may comprise Cadmium Tungstate, CdWO4. The scintillator may comprise Gadolinium oxysulfide, GOS. The scintillator may comprise a plastic scintillator such as BC-400, BC-404, BC-408, BC-498, or PEN. The scintillator may have a low phosphorescence in response to visible light exposure in order to reduce the amount of background noise picked up by the detector.

The scintillator product 300 of FIG. 3 may be used in a similar way to that described above in relation to FIG. 2, and a method of use is outlined here for completeness. In particular, according to the method a tissue sample is received from a subject, the tissue sample excised from the subject subsequent to a radiopharmaceutical being administered to the subject. The tissue sample is provided to a light tight enclosure of an imaging apparatus such as the imaging apparatus 100 described in detail above in relation to FIG. 1. The tissue sample is covered with the cloche 310 of the scintillator product 300. In this way, radiation emitted from within a few millimetres' depth of a surface of the sample that escapes the sample will impinge upon the scintillator product and may cause a scintillation event, detectable by the imaging apparatus. The imaging apparatus images the sample and cloche and to detect any scintillation events integrated over an exposure time and a determination is made as to whether or not the margins towards the exterior of the tissue sample are free of radiation emitting substances. In this way, one may determine whether or not the margin is tumour-free.

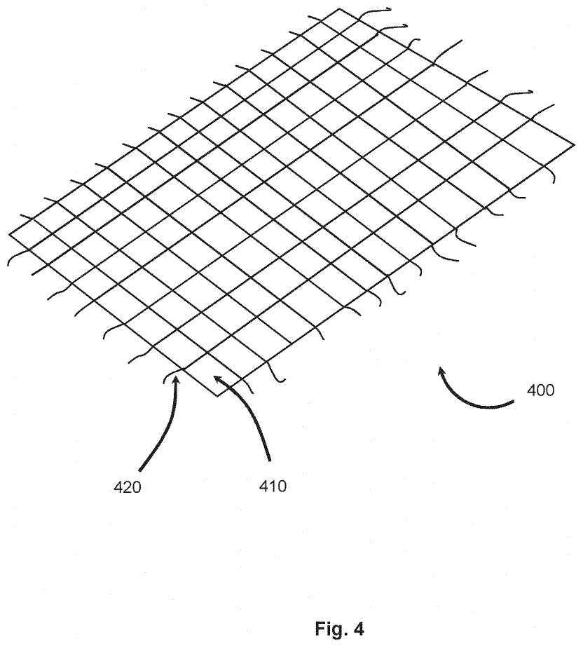

FIG. 4 illustrates a scintillator product according to another embodiment. In particular, FIG. 4 shows a scintillator product 400. The scintillator product 400 is for use in autoradiographic imaging of a tissue sample excised from a subject. The scintillator product 400 comprises a plurality of rigid scintillating elements, which in the present example comprise a plurality of rigid scintillating tiles 410 or plates. Each rigid scintillator tile 410 of the plurality of rigid scintillating tiles is configured to scintillate (luminesce), in use, in response to radiation from a radiopharmaceutical administered to the subject in advance of the excision. Each of the rigid scintillating tiles 410 is connected to at least one other rigid scintillating tile 410, in the present example by a connector such as a thread 420. Threads 420 run across the length and breadth of the scintillator product 400 connecting many of the rigid scintillator tiles 410. In this way, the scintillator product 400 forms a mosaic or array of interlinked rigid scintillator tiles 410. The tiles may be of any suitable shape. A tessellating shape may be used. The shape of the tiles may not all be the same.

Due to the connectors, or threads, each of the rigid scintillator tiles 410 is moveable with respect to another of the rigid scintillating tiles 410 such that, in use, the scintillator product is substantially conformable to the surface of the excised tissue sample, for scintillation in response to radiation emitted therefrom. A small gap may be provided between the tiles, or they may substantially face each other.

The scintillator product 400 of FIG. 4 may be used in substantially the same way as the scintillator products 200 and 300. The scintillator product may be used to cover, wrap, or drape over a surface of a tissue sample excised from a subject and, accordingly, will better follow the contours of the surface than can be achieved using a large rigid scintillator plate. Accordingly, any scintillation events detected by an imaging apparatus can be better mapped to a location on the surface of the sample, giving a higher spatial resolution and signal to noise ratio in the resultant image.

The tiles may be of any suitable size, for example each tile may have a length or a breadth of 1 cm, or 0.5 cm. The thickness of the tiles may be any suitable thickness, such as around 1 mm. As the tiles 510 themselves do not need to be flexible, the thickness of the tiles can be greater than the thickness of the scintillator product 200 of FIG. 1.

The tiles 410 may be coupled together in any suitable way. For example, although the tiles are linked by threads 420 in FIG. 4, they may be coupled through a hingeable connection. The scintillator product 400 may be formed as a single integral piece, in which the tiles 410 comprise a membrane such as that disclosed in reference to FIG. 1, and wherein the connections between the tiles are formed by thicker regions of membrane. In embodiments, the tiles may be embedded in or supported on a membrane carrier.

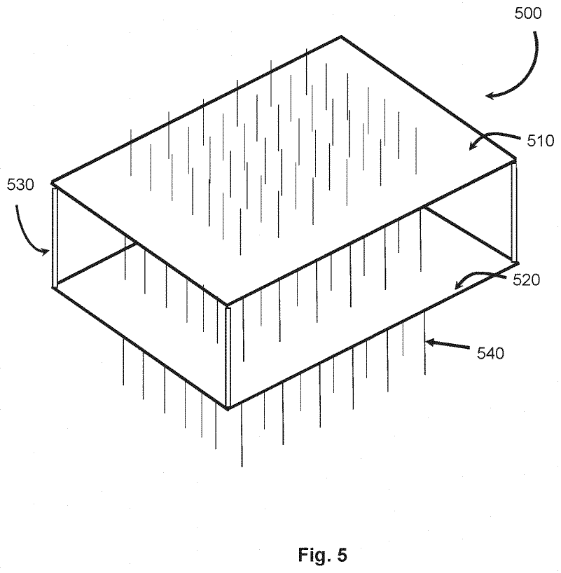

FIG. 5 shows a scintillator product 500 according to another embodiment. The scintillator product 500 is for use in autoradiographic imaging of a tissue sample excised from a subject. The scintillating product 500 comprises a body which in the present example comprises a first frame 510 and a second frame 520 connected via supporting columns 530. Frame 510 has a plurality of holes perforating its planar surfaces, and each hole of the frame 510 is aligned with a corresponding hole of the frame 520. The scintillator product 500 further comprises a plurality of rigid scintillating elements, which in the present example comprises a plurality of rigid rods or pins that may transmit light along their length, acting as a waveguide. Each elongate rod is slideable through a corresponding hole of the first frame 510 and the aligned hole of the second frame 520. Each rod 540, or at least material provided at an end face thereof, is configured to scintillate, in use, in response to radiation from a radiopharmaceutical administered to the subject in advance of excision of the sample. The scintillating rods 540 are moveable with respect to one another such that, in use, the scintillator product 500 is substantially conformable to the surface of the excised tissue sample, for scintillation in response to radiation emitted from the tissue sample. The scintillator product 500 comprises a matrix of scintillating rods 540, and each rod 540 is slideable through the matrix.

In use, the scintillator product 500 is placed over the excised tissue sample. Each scintillating rod 540 has a tip for contacting the excised tissue sample and the plurality of scintillating rods 540 thereby will conform to the surface of the sample. Each scintillating rod 540 is thereby associated with a corresponding location on the surface of the sample and, accordingly, any detected scintillation events can be mapped to a corresponding location on the surface of the sample.

By imaging the scintillator product 500 from above where it has been placed on the sample S, imaged scintillated light emitted at an end face of a scintillation rod 540 indicates a scintillation event from radiation from the margin region of the sample S where the scintillator rod 540 is resting on the sample S.

Although the scintillator product 500 has been shown with two frames 510 and 520 to aid in guiding each rod, the skilled person would appreciate that more or fewer frames may be present. The scintillator product may also comprise a plurality of stoppers in order to inhibit the movement of the scintillating rods beyond a certain distance, thereby preventing the scintillating rods from falling out of the scintillator product.

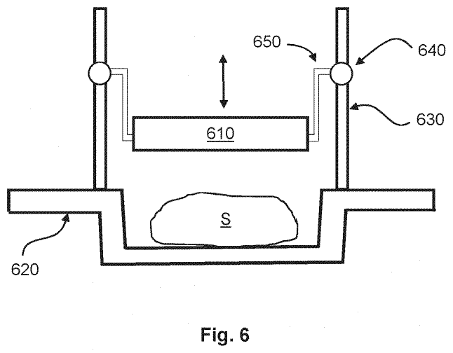

FIG. 6 shows a scintillator device 600 according to another embodiment. The scintillator device is provided with a tissue sample S. The scintillator device 600 is for use in autoradiographic imaging of a tissue sample excised from a subject, and may be used in conjunction with any suitable imaging apparatus, such as the imaging apparatus of FIG. 1.

The scintillator device 600 is for use with a tissue sample excised from a subject, wherein, in use, a surface of the tissue sample S is brought together with the scintillator device into a contiguous configuration in which the tissue sample is deformed such that a surface of the tissue sample and a surface of the scintillator device substantially conform to one another.

The scintillator device 600 comprises a scintillator sheet 610 configured to scintillate, in use, in response to radiation from a radiopharmaceutical administered to the subject in advance of the excision. The scintillator device 600 further comprises a retention mechanism (620, 630, 640, 650) for retaining the scintillator sheet 610 and the tissue sample together in the contiguous configuration.

In the embodiment shown in the figure, the scintillator device 600 comprises a tray in which the sample S can be positioned. The scintillator device 600 further comprises two arms 630 extending from the tray 620 and positioned so as to support the scintillator sheet 610 above the cavity of the tray into which the sample S is placed. The scintillator sheet 610 is held by supporting members 650 which are coupled to the arms 630 via a coupling mechanism 640, which may be adjusted so as to manoeuvre the scintillator plate 610 towards the sample S. For example, the coupling mechanism may comprise a locking wheel which can be loosened to substantially disengage the support members 650 from the arms 630.

When loading the scintillator device 600 with the sample S, the sample S may be placed in the cavity section of the tray 620. The coupling means can then be adjusted and the scintillator plate 610 can be lowered towards the sample until the scintillator plate 610 is in contact with a surface of the sample S. Light pressure between a rigid scintillator plate and the tissue sample S can cause the tissue sample to deform such that an enlarged surface area of the tissue sample S is pressed against/in contact with the scintillator plate 610. The coupling means 640 can then be adjusted (tightened) such that the retention mechanism (620, 630, 640, 650) retains/fixes the sample S and the scintillator sheet 610 together in a contiguous configuration. The scintillator device 600, once loaded with the sample S, can then be placed in a light tight chamber of an imaging apparatus for autoradiographic imaging.

The skilled person will appreciate that other structural arrangements for a scintillator device besides that shown in FIG. 6 are envisaged. The coupling means 640 may comprise any suitable coupling means, such as a clip, a screw, or a peg. The scintillator plate 610 may be a rigid plate.

Although in FIG. 6, a user may place the sample into the tray 620 and then position the scintillator plate 610 accordingly, the scintillator device may comprise a positioning mechanism for bringing the tissue sample S together with the scintillator device into the contiguous configuration. The positioning may be performed manually or automatically.

The scintillator device 600 may further comprise a transparent layer which, in the contiguous configuration, is positioned between the tissue sample S and the scintillator sheet 610. The transparent layer may prevent the sample from contaminating the scintillator plate 610, allowing the plate to be used again without substantially affecting imaging capabilities.

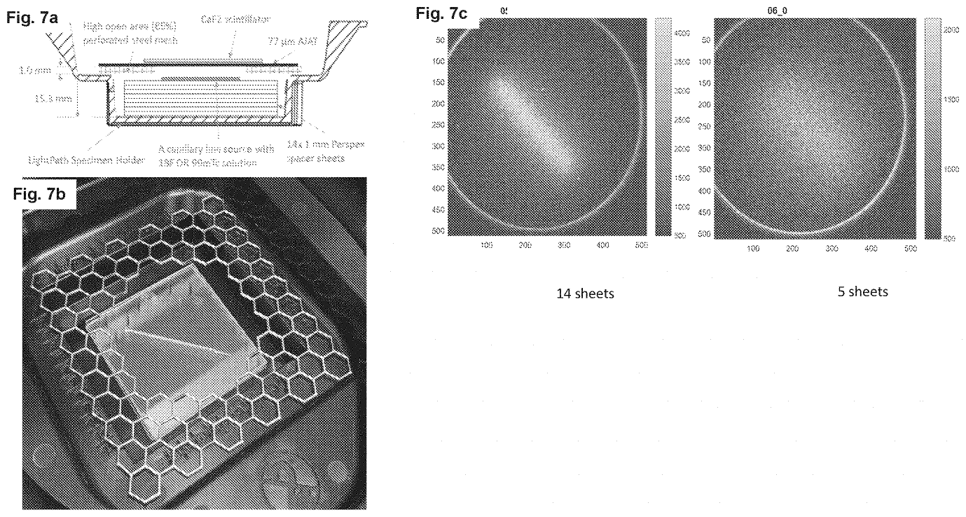

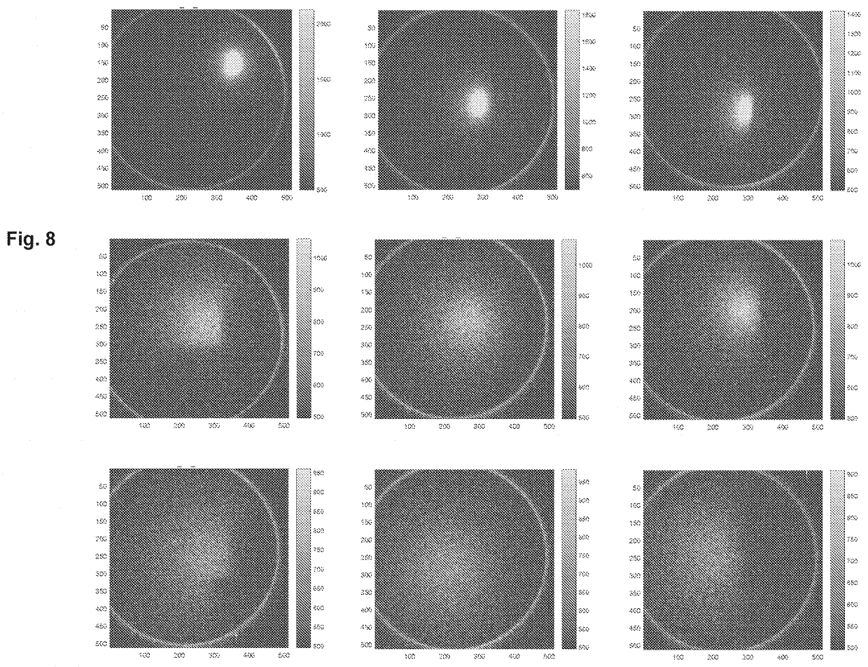

FIGS. 7 to 8 detail experiments performed by the inventors and show how both the spatial separation between a radioactive sample and a scintillating plate, and the angle of rotation of the scintillating plate relative to the radioactive sample, influence the spatial resolution of any low light luminescence images captured of the sample.

FIG. 7a is a schematic of the experimental set-up. A capillary line source with ¹⁸F (labelled 18F in the figure) was placed inside a specimen holder on top of a plurality of Perspex sheets by which the height of the capillary line source was established. The ¹⁸F acts as a radioactive source of beta radiation. A perforated steel mesh was placed above the capillary line source in order to support a rigid scintillation plate (comprising the scintillator CaF2) and also to provide a reference grid for determining the resolution of the resulting images. Inside a light tight imaging apparatus, the resultant scintillation events were detected using a cooled emCCD. FIG. 7b is a photograph of the capillary line source on top of several Perspex sheets and with the steel mesh clearly visible.

FIG. 7c shows how the spatial resolution of the resultant images varies with the height difference between the samples. In the first image of FIG. 7c, 14 sheets of 1 mm thick Perspex are placed beneath the capillary line source; in the second image of FIG. 7c, 5 sheets of 1 mm thick Perspex are placed beneath the capillary line source. As is shown in the Figure, on the left hand side where 14 Perspex sheets are beneath the sample and the sample is closer to the scintillating plate, the spatial resolution and signal to noise ratio in the image of the resulting distribution of scintillation events is relatively high. In contrast, on the right hand side, where only 5 Perspex sheets are beneath the sample and the sample is further away from the scintillating plate, the spatial resolution and signal to noise ratio in the image of the resulting distribution of scintillation events is low. Thus the spatial resolution and signal strength is much greater when more Perspex sheets are placed beneath the sample (i.e. when the radioactive source is closer to the scintillator).

FIG. 8 shows how the spatial distortion varies for different distances and different angles between the rigid scintillating plate and an ¹⁸F source. In particular, the images of the left-most column were taken with the scintillating plate at a zero degree angle relative to the horizontal above the source; the images of the middle column were taken with the scintillating plate at a 30 degree angle of the scintillating plate relative to the source; and the images of the right-most column were taken with the scintillating plate at a 60 degree angle relative to the horizontal above the source. The images of the top row were taken with a distance between the rigid scintillator plate and the source of 2 mm; the images of the middle row were taken with a distance between the rigid scintillator plate and the source of 8.5 mm; and the images of the bottom row were taken with a distance between the rigid scintillator plate and the source of 15 mm. The change in the presentation/incidence angle of the surface of the scintillator away from normal to the source of the radiation can lead to a distortion of the resulting imaged distribution of scintillation events. FIG. 8 clearly shows that when a rigid scintillator plate is used, the distance between the plate and the source, and the angle of the plate relative to the source, greatly affect the spatial resolution of the resultant images obtained of scintillation events that occur.

FIGS. 7 and 8 illustrate that effective margin assessment can be performed by digital autoradiography of an intact tissue sample during surgery using a scintillator product of the examples of the present disclosure to conform closely to the surface of the tissue sample, and by integrating an image of the scintillation events in a low light environment using a sensitive, low light camera to reveal an autoradiographic image of emissions from the margin of the sample.

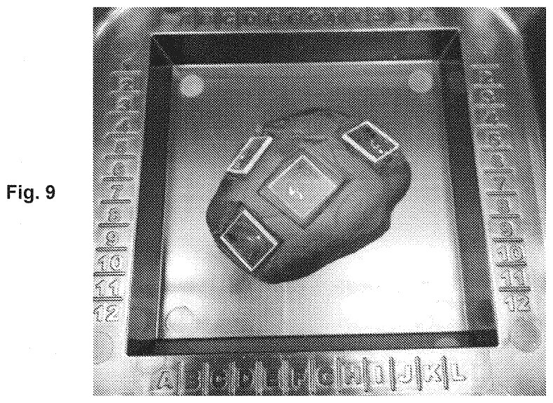

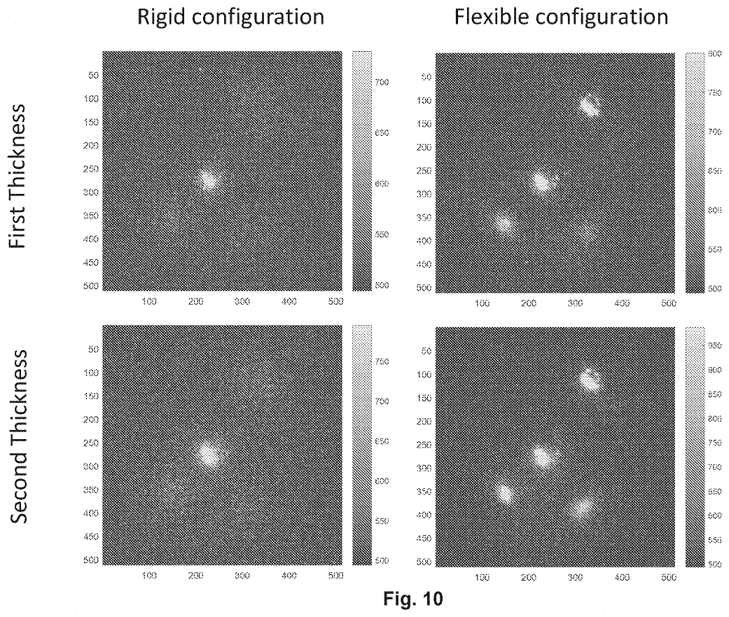

FIGS. 9 and 10 illustrate that a scintillator product able to conform the surface of a tissue sample provides a better signal than a rigid scintillator plate suspended closely above the sample. FIG. 9 shows a breast mimicking intact tissue phantom provided, in four different locations, with an amount of the medical radioisotope technetium-99m, ^{99m}Tc. The radioisotope was provided at one location at the top at the uppermost surface of the sample, and at three locations to the side, lower down the sample as it rested on the sample tray. Autoradiographic images are shown of the tissue sample using a rigid scintillator suspended closely above the sample (this arrangement is not shown in FIG. 9), and using a scintillator arrangement provided by the ‘flexible’ scintillator products of the present disclosure. As shown in FIG. 9, To mimic the arrangement of a ‘flexible’ or conformable scintillator product in accordance with the examples, four individual scintillator plates or ‘tiles’ were arranged in close contact with the locations of the radioisotope, as shown, closely facing and resting on the surface of the tissue phantom.

FIG. 10 shows the contrast between the rigid configuration of the scintillator and a flexible configuration of the scintillator, and also the different signals received using different thicknesses of the scintillator. The top row shows images relating to a scintillator having a first thickness of a few microns. The bottom row shows images relating to a scintillator having a second thickness double that of the first thickness. As shown on the left, the rigid suspended scintillator, for both thicknesses, provides a detectable signal above the noise for only the radioisotope dose provided at the top of the sample. Scintillation events from the other three radioisotopes do not provide scintillation events having a distribution on the surface of the rigid scintillator to provide any useful spatial resolution, or signal strength above the noise level sufficient to enable detection of the radioisotope at the surface of the tissue phantom. In contrast, as shown on the right, the autoradiographic image of the scintillator tiles arranged as in a ‘flexible’ configuration shows good spatial resolution and signal strength above the noise background of all four radiation sources, meaning that a surgeon can effectively use the scintillator products of the present disclosure to perform margin analysis of intact samples during surgery. This enables surgeons to identify and locate abnormal tissue at the margin of samples during surgery, and excise further tissue from the patient to increase the confidence that all abnormal tissue is removed, and to reduce the likelihood of reoperation. The thicker scintillator (bottom row of FIG. 10) provided a stronger signal than the thinner scintillator (top row of FIG. 10).

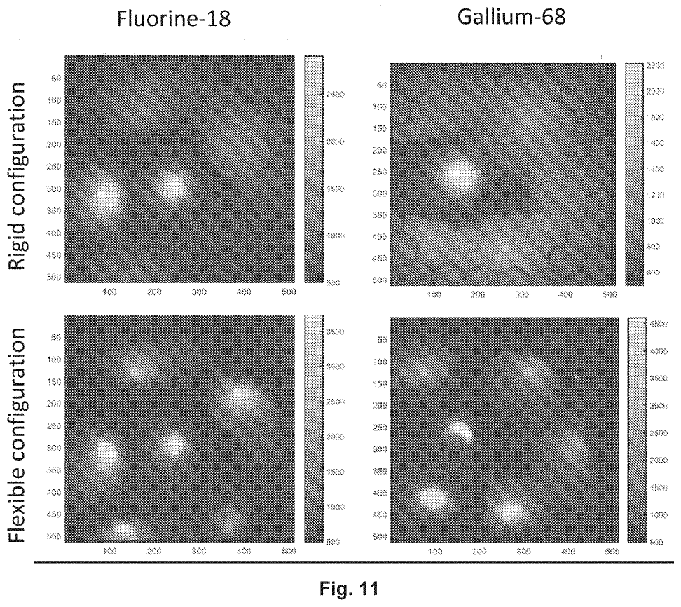

The images of FIG. 10 were generated using a Technetium-99m source. FIG. 11 shows the contrast between a rigid configuration and a flexible configuration when two other sources are used, namely Fluorine-18 and Gallium-68. For both sources, the flexible configuration provides a far stronger signal and better spatial resolution than the rigid/flat configuration, in which the surface of the phantom/sample and the surface of the suspended scintillator plate do not conform to one another.

Variations of the described embodiments are envisaged, for example, the features of all the described embodiments may be confined in any way.

The radioisotope used may be any of the radioisotopes referred to above or any other suitable radioisotope. The radioisotope may be a positron emitter such as 11C, 13N, 15O, 18F, 44Sc, 62Cu, 64Cu, 68Ga, 76Br, 86Y, 89Zr, or 124I.

The radioisotope may be a pure or impure electron emitter such as ³H, ¹⁴C, ³⁵S, ³²P, ³³P, 35S, 59Fe, 99mTc, 111In, 125I, 137Cs, 90Y, 177Lu, 153Sm, ¹³¹I, ⁵⁹Fe, ⁶⁰Co, ⁶⁷Cu, ⁸⁹Sr, ⁹⁰Sr, ⁹⁰Y, ⁹⁹Mo, ¹³³Xe, ¹³⁷Cs ¹⁵³Sm, ¹⁷⁷Lu, or ¹⁸⁶Re.

The subject from which the tissue sample is excise may be any suitable subject such as a human or an animal.

In order to suppress noise from background gamma radiation, a scintillator product as described herein may comprise multiple scintillator layers, which may have different thicknesses. A hatched scintillator material may also be used.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A flexible scintillator product for use in autoradiographic imaging of a tissue sample excised from a subject, the flexible scintillator product comprising:
a membrane provided with a scintillator,
the scintillator being configured to scintillate, in use, in response to radiation from a radiopharmaceutical administered to the subject in advance of the excision; and
wherein the membrane is freely conformable such that, in normal use, the scintillator product is wrappable around a surface of the excised tissue sample, for scintillation in response to radiation emitted therefrom.

2. A scintillator product according to claim 1, wherein the flexible scintillator product has a thickness of less than or equal to 3 millimetres.

3. A scintillator product according to claim 2, wherein the flexible scintillator product has a thickness of less than or equal to 1 millimetre.

4. (canceled)

5. A scintillator product according to claim 1, wherein the membrane material is the scintillator.

6. A scintillator product according to claim 5, wherein the membrane comprises one of BC-400, BC-404, BC-408, BC-498, or polyethylene naphthalate, “PEN”.

7. A scintillator product according to claim 1, wherein the scintillator is integral to the membrane.

8. A scintillator product according to claim 1, wherein the scintillator is provided as a layer on the membrane.

9. A scintillator product according to claim 1, wherein the scintillator comprises ZnS:Ag, ZnCdS:Ag, YSO:Ce CsI:Tl, YAG:Ce, Y2O2S:Tb, or ZnSe:O.

10. A scintillator product according to claim 1, wherein the scintillator product is at least 10% transparent to electromagnetic waves having a wavelength in the range of 400 nm to 700 nm.

11. (canceled)

12. A scintillator product according to claim 1, wherein the scintillator has a peak emission wavelength in the range of 400 nm to 500 nm.

13. A scintillator product according to claim 1, wherein the scintillator has a peak emission wavelength in the range of 500 nm to 600 nm.

14. A scintillator product according to claim 1, wherein the scintillator product further comprises a pattern, the pattern printed on the membrane and visible under illumination.

15. A scintillator product according to claim 1, further comprising a transparent dielectric or reflective coating for reducing scattering losses.

16.-34. (canceled)

35. Apparatus for autoradiography of a tissue sample excised from a subject, the apparatus comprising:
a scintillator product according to claim 1 provided with a tissue sample excised from a subject; and
detection apparatus comprising:
an enclosure, the excised tissue sample provided with the scintillator product being arranged inside the enclosure; and
a detector arranged to detect scintillated light from within the enclosure from the scintillator product.

36. A method for analysing a tissue sample excised from a subject, the method comprising:
providing a scintillator product according to claim 1 to the tissue sample; and
detecting scintillated light from the scintillator product, the scintillated light generated in response to radiation emitted from the tissue sample.

37. A method according to claim 36, the method further comprising enclosing the tissue sample with the scintillator product or the scintillator device in a light tight enclosure.

38. A method according to claim 36, wherein detecting scintillated light comprises detecting scintillated light using an electron multiplying charge coupled device, emCCD.

39. A method according to claim 36, wherein detecting scintillated light comprises detecting scintillated light using a scientific complementary metal-oxide-semiconductor, sCMOS, detector.

40. A method according to claim 36, the method further comprising capturing a photographic image of the tissue sample.

41. A method according to claim 40, further comprising mapping detections of the scintillated light to corresponding locations on the photographic image.

42.-49. (canceled)
